(19) United States
(12) Patent Application Publication
(10) Pub. No.: US 2008/0108787 A1
(43) Pub. Date: May 8, 2008

(54) Purification of Insulin-Like Material by Reverse Phase Chromatography

(76) Inventors: Maharaj K Sahib, Aurangabad (IN); Edupuganti B Raju, Aurangabad (IN); Sivaraman Subramaniam, Salem (IN); Vennapusa R Reddy, AndhraPradesh (IN)

Correspondence Address: BIO INTELLECTUAL PROPERTY SERVICES (BIO IPS) LLC, 8509 KERNON CT., LORTON, VA, 22079, US

(21) Appl. No.: 11/597,430

(22) PCT Filed: May 24, 2004
(86) PCT No.: PCT/IB04/01869

§ 371 Date: Jul. 20, 2007

(51) Int. Cl. C07K 1/16

(52) U.S. Cl. 530/305

(57) ABSTRACT

This invention describes processes for purification of insulin or insulin-like material by reverse phase chromatography by using polystyrenic resins as the chromatographic materials. in particular the present invention describes processes for the purification of a particular insulin-like material from chemically and structurally similar contaminants.

### BACKGROUND

There are several chromatographic methods available for the isolation and purification of proteins, the choice of method/s determined by the properties of the protein to be isolated, as well as the nature and degree of contaminants present in the protein source.

A protein source is usually a complex mixture, comprising the protein to be isolated, as well as non-essential contaminant proteins and polypeptides. While it is relatively easy to get rid of contaminants with properties very different from that of the protein of interest, separation of a protein from contaminants of similar, or near identical, properties, is usually a more difficult task. For example, proteins/polypeptides with molecular weight or surface charge very different from the protein of interest may be routinely separated by gel-filtration or ion-exchange chromatography. On the other hand, the chromatographic eluant fraction, containing the protein of interest, may not be a homogenous solution, and may often contain smaller quantities of contaminants with properties very similar to that of the protein of interest. Such contaminants could be degradation products, analogs, protein-expression and secretion artifacts, or side products of a chemical reaction (such as derivatization) of the protein of interest. Quite apart from the nature of the contaminants present in the protein source, another factor that determines the choice of purification techniques is the structural stability of the protein. The activity of a protein can be effected by its stability, and protein stability can in turn be effected by relatively small changes in the solvent composition, including pH, salt concentration, buffer, temperature etc. The choice of the purification process must be such as to have a minimal effect on the protein's structural stability. Strongly hydrophilic resins have often been the resins of choice for the purification of proteins, since these resins, by maintaining an aqueous environment, have minimal effects on a protein's structural integrity. However, hydrophilic resins have certain disadvantages. These include an increased susceptibility to even medial back-pressure and greater difficulty in removing non-specific adsorption. Alternative purification procedures involve the use of more hydrophobic resins. Specifically, reverse-phase high-performance liquid chromatography has been frequently used for purification, because it can efficiently separate even closely related protein impurities. The resins commonly used in reverse phase chromatography are usually silica based, in which lipophilically modified silica gel is the stationary phase of the chromatography. Examples of such resins include C-4, C-8 or C-18 modified silica resins, in which n-alkyl hydrocarbon ligands are attached to silica resins.

U.S. Pat. No. 5,780,593 describes a method for isolating biomolecules by ion exchange chromatography in which post-loading, the bound biomolecules are eluted by an eluant comprising charge neutralizing acid or base, that can transform the ion exchange groups from the charged form to the uncharged form.

U.S. Pat. No. 5,101,013 describes a process for the isolation of basic proteins, obtained by enzymatic reaction of proinsulin, by strong acid cation exchange chromatography. In particular the patent specifies that the proteins are eluted with a 10-50% by volume C₁-C₄ alkanol solution and at a pH 2.5-5.0.

U.S. Pat. No. 5,977,297 describes the use of a pressure-stable acidic cation exchange chromatography for the isolation of insulin.

U.S. Pat. No. 6,451,987 describes a process for the purification of a peptide from related impurities by cation exchange chromatography. Specifically, it claims the use of an organic modifier containing buffer for the removal of impurities bound to the column post-loading.

U.S. Pat. No. 6,265,542 claims a process for purifying a polypeptide by reversed-phase liquid chromatography using an elution buffer containing hexylene glycol.

U.S. Pat. No. 5,094,960 describes a process for removing lipid soluble compounds from biological material (for example blood plasma) by hydrophobic interaction chromatography column containing C-6 to C-24 resin. In this process the lipid soluble compound is retained in the column, while rest of the biological material passes through the column.

U.S. Pat. No. 4,616,078 describes a process for the isolation of proinsulin-like material using a reverse phase macroporous acrylate ester copolymer resin. The process conditions include, eluting the bound proinsulin-like material with an eluant at pH 8-11 and having 10-30% by volume organic solvents—acetone, acetonitrile and a combination of the two.

U.S. Pat. No. 5,245,008 describes a process for the purification of insulin and insulin derivatives on lipophilically modified silica gel using a buffer containing organic solvents and alpha-amino acids or betaines, the pH of the buffer being one pH unit above or below the isoelectric point of the insulin or its derivatives.

U.S. Pat. No. 5,621,073 describes a process for the purification of insulin on a lipophilically modified silica gel using buffers containing zwitterions and organic solvents comprising acetone or acetonitrile.

In the present invention we use polystyrenic resins for the purification of insulin-like materials from solutions that contain impurities, including closely related ones like polypetides. Specifically we describe the use of polystyrene-divenyl-benzene resins for the purification of insulin or insulin-like materials. Polystyrenic resins provide several advantages over silica based ones due to their stable polymeric structure. Chemical stability includes greater pH stability in that, whereas silica based gels are stable in the pH range 2-7, polystyrenic resins have a much wider pH range stability (2-14). This allows much greater resolution of polypeptides and proteins with a higher proportion of polar amino acid residues, especially on the polypeptide surface. In addition, the wider pH stability range, permits the use of more extreme pH-cleaning-solutions. In the case of silica based resins, the use of pH greater then 8 for the post-elution cleaning-in-place results in the cleavage of the hydrophobic arm from the silica matrix. There is thus greater risk of “ligand-leaching”. This either precludes traditional post-elution sanitation, necessary to kill bacteria, altogether, or if extreme pH cleaning-in-place is carried out, reduces the shelf-life of the chromatography resin. Polystyrenic resins, on the other hand, permit the use of strong acid or base solutions for cleaning-in-place. Thus the same resin can be used for several cycles of protein purification, a highly cost saving measure.

In the present invention, the term insulin-like material as used herein includes insulin of human and non-human origin, such as those of porcine or bovine origin. They also include precursors such as proinsulins and preproinsulins, recombinant insulins, insulin derivatives or polypeptides that perform roles similar that of insulin. Insulin derivatives can be obtained by chemical or enzymatic reactions, for example InsulinB-30(threonine)-t-butyl ester-t-butyl ether obtained by reacting des(30)miniproInsulin with threonine-butyl ester-butyl ether in the presence of trypsin. The term also encompasses analogs in which one or more amino acids may be changed, replaced, deleted or added, as well as derivatives of these analogs obtained by chemical or enzymatic reactions.

### DESCRIPTION OF INVENTION

The present invention describes a process for the purification of insulin-like materials.

The production of insulin by recombinant DNA methods is a multi-step process. Starting with the gene encoding the insulin polypeptide, the process involves transforming a suitable microbial host with the vector carrying the gene, followed by subjecting the transformed host to conditions that induce it to express the insulin polypeptide. The polypeptide so expressed is either retained inside the host cell or secreted into the medium. Following expression, the polypeptide is then isolated from the culture medium in a highly purified form. This “isolation” process, often described as “down stream processing” (DSP), is usually a multi-step process that includes subjecting the polypeptide to chemical and enzymatic reactions and several chromatographic steps to gradually purify the polypeptide and/or its derivatives. With each chromatographic step, while most of the impurities, unrelated physico-chemically to the insulin-like material, is removed quite easily, the purified insulin-like material may still be contaminated by structurally related impurities, as well as impurities that are a result of chemical and/or enzymatic side reactions, or unreacted reactants. Further purification of the insulin-like material may then be necessary. The following example would serve only to illustrate the process. Insulin may conveniently be expressed as a precursor polypeptide B(1-29)-A(1-21) (also depicted as des(B30)miniproInsulin), in which the amino acid 29 of the B chain is connected through a peptide linkage with the amino acid 1 of the A chain. Such a polypeptide may be conveniently expressed in a recombinant host, such as yeast, in very large amounts. The expressed polypeptide is usually subjected to an initial purification step to remove a large proportion of impurities that are present in the medium of the expression host. This is then followed by conversion of the insulin precursor to native insulin (depicted as B(1-30):::A(1-21)), a peptide that has the amino acid threonine at position 30 in the B chain, two inter-chain disulfide bonds between the B and A chains and one intra-chain disulfide bond within chain A. The conversion to native insulin is carried out in two steps. The first step consists of reacting insulin precursor to threonine-butylester-butylether in the presence of trypsin to obtain InsulinB-30(threonine)-t-butyl ester-t-butyl ether. The latter is then hydrolyzed in the presence of tryptophan to obtain native insulin—B(1-30):::A(1-21). (Note: InsulinB-30(threonine)-t-butyl ester-t-butyl ether indicates that amino acid threonine is present at position 30 of the B chain of insulin, with the -t-butyl ester-t-butyl ether moiety is mostly attached to the carboxyl group of the said threonine.). The entire process may be depicted schematically as:

### B(1-29)-A(1-21) expression→Initial purification→B(1-29)-A(1-21)

### B(1-29)-A(1-21)+Threonine-butylester-butylether+Trypsin→InsulinB-30(threonine)-t-butyl ester-t-butyl ether

### InsulinB-30(threonine)-t-butyl ester-t-butyl ether+Tryptophan+Trifluoroacetic acid →B(1-30):::A(1-21).

The “initial purification” usually removes most of the impurities—protein, polypeptide, peptide etc.—especially those that differ considerably in physico-chemical properties from the insulin precursor. However, the fraction of the eluate from the initial purification step, that contains the insulin precursors, would nevertheless contain impurities with properties similar to that of the insulin precursors (such as degradation or other artifacts of expression and secretion of insulin polypeptide by the recombinant host). In addition, following the reaction of the insulin precursor with threonine-butyl ester-butyl ether and trypsin, the product solution containing InsulinB-30(threonine)-t-butyl ester-t-butyl ether, would also contain some unreacted insulin precursors, as well as peptides generated by trypsin activity. Likewise, when insulinB-30(threonine)-t-butyl ester-t-butyl ether is hydrolyzed in the presence of tryptophan (trifluoroacetic acid) to give native insulin B(1-30):::A(1-21), the product solution would contain some unreacted Insulin(B-30 threonine t-butyl ester-t-butyl ether) as well. Thus at the end of any of the above steps, it would be desirable to remove the corresponding undesirable impurities. In the present invention we describe a process for the purification of insulin-like materials from solutions that contain such impurities. Specifically we describe the use of polystyrene-divenyl-benzene resins for the purification of insulin-like materials. The purification process can, for instance, be used for the isolation of Insulin(B-30 threonine t-butyl ester-t-butyl ether) after the threonine-butylester-butylether/trypsin reaction. Such an isolation would be highly desirable, since purified Insulin(B-30 threonine t-butyl ester-t-butyl ether) would be more efficiently hydrolyzed to native insulin. Likewise, the process described in the present invention could also be used for the purification of native insulin (B(1-30):::A(1-21)) after the hydrolysis step, as well as the insulin precursor (B(1-29)-A(1-21)) prior to the threonine-butylester-butylether/trypsin reaction.

We first describe below a process for the expression of insulin precursor by recombinant DNA technology. This is then followed by examples illustrating the down stream processing steps that result in the isolation and purification of native insulin. The descriptions and examples only serve to illustrate the present invention. It should however be understood that they do not in any way restrict the scope of the invention.

### Construction of the Recombinant Vector Carrying the Insulin Polypeptide Gene

In the description below, the sequence of the polypeptide expressed by the recombinant host (yeast strain Hansenula polymorpha) is provided in the Seq ID1. Seq ID2 is the DNA sequence corresponding to that of amino acid sequence in Seq ID1.

In the seq ID1, the peptide region from amino acid 1 to 85 is the mating factor alfa (MFα) leader peptide from Saccharomyces cerevisiae that is required for the secretion of the expressed product into the extracellular medium. The MFα leader sequence carries a Kex2 protease site and is removed by yeast processing enzyme Kex2 protease just prior to secretion. Thus the polypeptide that is eventually secreted is B(1-29)-A(1-21) (insulin “precursor”) where B(1-29) is the B-chain peptide from amino acid 1 to amino acid 29 of the “native” insulin B chain and A(1-21) is the A-chain peptide from amino acid 1 to amino acid 21 of the “native” insulin A chain. In B(1-29)-A(1-21), the amino acid 29 of the B chain is connected by means of a peptide bond to amino acid 1 of the A chain. B(1-29)-A(1-21) corresponds to amino acid sequence stretch 85-135 in Seq ID1. B(1-20)-A(1-21) may also be depicted as des(B30)miniproInsulin.

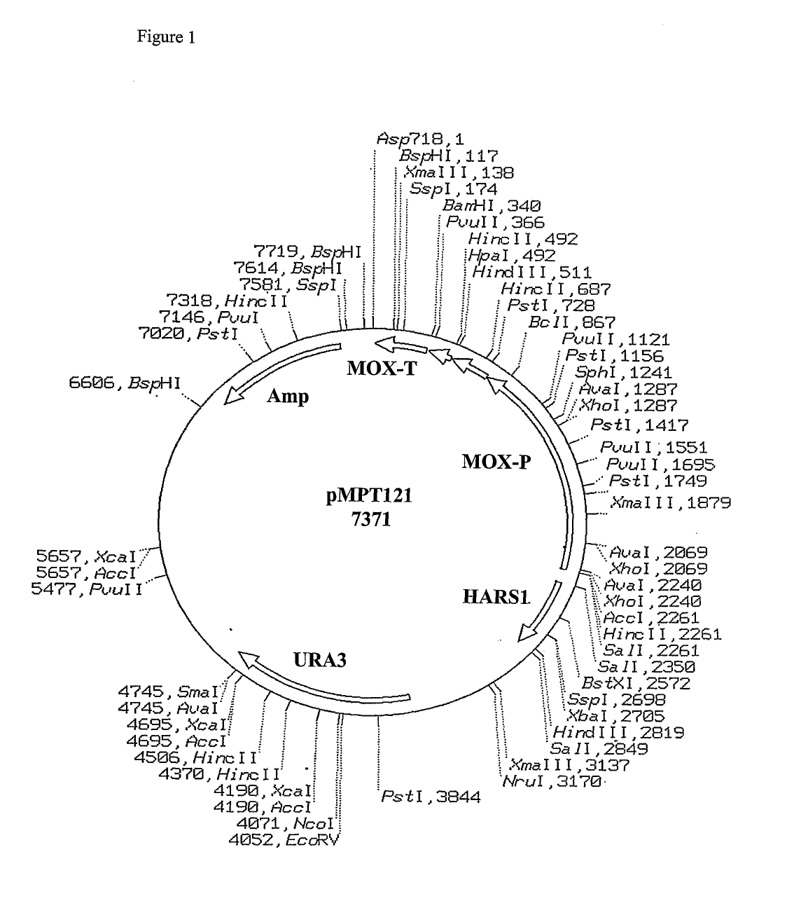

The gene, as represented in Seq ID2, was constructed taking into account the codon usage by the host (in the present case, the yeast strain Hansenula polymorpha). The DNA construct comprising the gene possess cleavage sites for two restriction enzymes—EcoRI and BamH1—on either sides of the gene. The DNA construct so obtained was cloned into the site created by EcoRI and Bam-H1 restriction enzyme digation of the plasmid expression vector pMPT121 (FIG. 1) by methods well known to those of ordinary skill in the art (“Molecular Cloning: A Laboratory Manual” by J. Sambrook, E. F. Fritsch and T. Maniatis, II edition, Cold Spring Harbour Laboratory Press, 1989) and transformed into E.coli hosts by methods also well known to those skilled in the art (“Molecular Cloning: A Laboratory Manual” by J. Sambrook, E. F. Fritsch and T. Maniatis, II edition, Cold Spring Harbour Laboratory Press, 1989)

The pMPT121 plasmid expression vector is based on a pBR322 plasmid and contains the following elements:
standard E. coli pBR322 skeleton including E. coli origin of replication (ori).
ampicilin resistance gene for selection of transformed E. coli.
URA3 (orotidine-5′-phosphate decarboxylase) auxotrophic selective marker gene complementing the auxotrophic deficiency of the host—Hansenula polymorpha
H. polymorpha Autonomously Replicating Sequence (HARS).
an expression cassette containing the MOX promoter and the MOX terminator for insertion of the gene construct and controlling the expression of the cloned heterlogous polypeptides in the said yeast strain.
   Individual E.coli clones carrying the recombinant plasmids were cultured and the plasmids isolated by methods well known in the art (“Molecular Cloning: A Laboratory Manual” by J. Sambrook, E. F. Fritsch and T. Maniatis, II edition, Cold Spring Harbour Laboratory Press, 1989). The isolated recombinant plasmids were then confirmed to be carrying Seq ID2, by DNA sequencing.

### Transformation of a Yeast Strain with the Recombinant Vectors Carrying the Insulin Polypeptide Gene

The recombinant plasmids obtained as above were transformed into the yeast strain H. polytizorpha (which is an ura3 auxotrophic mutant deficient in orotidine-5′-phosphate decarboxylase) by methods known in the art (Hansenula polymorpha: Biology and Applications, Ed. G. Gellissen. Wiley-VCH, 2002). The resulting recombinant clones were then further used for the expression of the polypeptide

### Expression of the Insulin Polypeptide in Yeast

The yeast transformants thus obtained were used for the expression of the polypeptide. The expression conditions were:

a)Preculture:

Single clones, each carrying the expression vector containing the DNA sequences encoding the polypeptide (viz. Seq ID2 corresponding to Seq ID1) were inoculated into 100 ml pre-sterilised YNB/1.5% glycerol medium in a 500 ml shake flasks with baffles. The composition of the YNB/1.5% is 0.28 g yeast nitrogen base, 1.0 g ammonium sulfate, 1.5 g glycerol and 100 ml water. The cultures were incubated for about 24 h at 37° C. with 140 rpm shaking until an O.D₆₀₀ of 3-5 is reached

b) Culture:

450 ml of Pre-sterilised SYN6/1.5% glycerol media in 2000 shake flasks with baffles were inoculated with 20-50 ml of each of the above preculture. The cultures were then incubated for 36 h at 30° C. and 140 rpm. The composition of the SYN6/1.5% glycerol medium is NH₄H₂PO₄—13.3 g, MgSO₄×7H₂O—3.3 g, NaCl—0.3 g, glycerol—15.0 g, in water 1000 ml. The media was further supplemented with CaCl₂, microelements, vitamins and trace elements.

c) Fermentation:

10 L of SYN6 medium was autoclaved in a fermentor for 20 min. at 121° C. After autoclaving, the temperature, pH, aeration and agitation were set to the desired values (pH to 4.0, agitation to 400 rpm, aeration to 1 vvm). The fermentor was inoculated with the seed culture and fermentation continued for additional 5 days maintaining dissolved oxygen concentration (DO) above 20%. Samples were collected at regular intervals and at the end of fermentation to check for growth, product concentration, pH and state of the cells.

### Isolation, Purification and Conversion of the Insulin Precursors to “Native” Insulin.

At the end of the fermentation cycle, culture containing the secreted insulin precursor (B(1-29)-A(1-21)) was clarified by centrifugation and isolated by cation exchange chromatography.

### EXAMPLE 1

### Cation Exchange Chromatography.

A Chromatography column of about 100 mm×50 cm dimensions was packed with 200 ml cation exchange SP-Sepharose fast flow (Pharmacia) resin. The column was regenerated with 0.5N NaOH, washed with deionised water and then equilibrated with 20 mM citrate buffer at pH 4.0. The supernatent obtained after clarification was diluted two fold with 20 mM citrate buffer, and applied on to a cation exchange column at pH 4.0 and flow rate of 200 cm/h. The column was then washed with 20 mM citrate buffer (5 Column Volumes) at a flow rate of 200 cm/h, and the bound polypeptides eluted with 100 mM tris HCl, pH 7.5 buffer, at a flow rate of about 100 cm/h.

### EXAMPLE 2

### Isoelectric Precipitation.

Single chain insulin precursors obtained as eluate from cation exchange chromatography was quantified and treated with an equal quantity of solid zinc chloride (viz. 1:1 w/w as that of insulin precursor). The pH was adjusted to about 6.0 with HCl to precipitate the insulin precursors. The precipitated precursor was allowed to settle at 8° C. for about 12 hours, followed by centrifugation and drying to obtain dry insulin single chain precursors.

### EXAMPLE 3

### Transpeptidation: Conversion of the Insulin Precursor Polypeptide, B(1-29)-A(1-21) to InsulinB30(threonine)-t-butyl-ester-t-butyl-ether

About 3.0 g of single chain insulin precursors obtained in example 2 were dissolved and incubated at 12° C., in a reaction mixture containing 23.6 ml of dimethyl sulfoxide/methanol (50/50 v/v), 15.0 g of L-threonine-t-butylester-t-butyl ether, 14.4 ml milliQ water and 300 ul of acetic acid. Prechilled solution containing 150 mg of bovine pancreatic trypsin dissolved in 2.55 ml of 50 mM calcium acetate, 0.05% acetic acid, pH adjusted to 7.3 with acetic acid or ammonia was then added. The reaction mixture was incubated at 12° C. for about 4-8 hours. Progress of the reaction was monitored by analytical RP-HPLC. After achieving >80% conversion of insulin precursors to InsulinB30(threonine)-t-butyl-ester-t-butyl-ether, reactions were quenched by reducing the pH to 3.0 with 1N HCl. InsulinB-30(threonine)-t-butyl ester-t-butyl ether) may then be purified to remove the unreacted precursors and other undesired side products, such as desB30(Thr)insulin and des(23-30)octapeptide insulin prior, to the hydrolysis step (see example 5 below). Alternatively, InsulinB30(threonine)-t-butyl-ester-t-butyl-ether may be isolated by isoelectric precipitation and drying, followed by hydrolysis of the impure esters and then purification to obtain pharmaceutical grade insulin in a single step purification process (see example 7 below).

### EXAMPLE 4

### Hydrolysis.

About 180 mg of lyophilized insulinB-30(threonine)-t-butyl ester-t-butyl ether was hydrolyzed to “native” insulin in a 100 ml round bottom flask by dissolving it in anhydrous trifluoroacetic acid (TFA) at a concentration of 10 mg insulin derivative per ml TFA, in presence of 0.5 mg tryptophan per ml of TFA. The reaction mixtures were kept at 25° C. for 20 min. TFA was removed from the reaction mixture under reduced pressure in a Buchi rota evaporator and the residue mass resuspended in 20 ml 1% acetic acid (v/v). Native insulin so obtained may then be further purified as described below (see examples 6,7).

### Purification of InsulinB-30(threonine)-t-butyl ester-t-butyl Ether and Native Insulin on Polystyrenic Resins

During the conversion of Insulin polypeptide to insulinB-30(threonine)-t-butyl ester-t-butyl ether by transpeptidation with L-threonine-t-butyl ester-t-butyl ether and trypsin, several non-specific products, a result of side/non-specific reactions, are produced. These include, des(B30)threonine insulin and desoctapeptide B(22-30) human insulin generated by proteolysis, peptides generated from trypsin activity, denatured auto digested trypsin products etc. These contaminants, along with less characterized insulin variants generated during fermentation, such as acylated insulins, may be removed in a single step by purification with reverse phase chromatography using polystyrene divenylbenzene resins. Likewise, native insulin (B(1-30):::A(1-21)) obtained by hydrolysis of InsulinB-30(threonine)-t-butyl ester-t-butyl ether is also contaminated with several non-specific products. Purification of native insulin may also be carried out by reverse phase chromatography using polystyrene divenylbenzene resins. Examples of include Amberchrom CG 300S, Amberchrome CG 300XT etc (available at Rohm and Haas co.). Polystyrenic based reverse phase resins are superior to conventional lypophylically modified silica supports in having greater mechanical strength and much wider pH range stability. The latter is an especially important feature as it permits the use of extreme pH solutions for the post-elution cleaning-in-place.

### EXAMPLE 5

### Purification of InsulinB-30(threonine)-t-butyl ester-t-butyl Ether) on Polystyrenic Resins

At the end of the conversion reaction (see example 3) trypsin activity is first quenched by reducing the reaction pH to 3.0 with 1N HCl and diluted 2 fold with deionised water. A medium pressure chromatography column (26 mm×500 mm) is packed with 150 ml Amberchrome CG 300sd reverse phase chromatography resin (Rohm and Haas). The resin is regenerated with 0.5N NaOH, washed with water and 80% isopropanol containing 0.1% TFA and then equilibrated with buffer A (5CVs). 3 g of impure Insulin(B-30)-threonine-t-butyl ester-t-butyl ether is applied to the column at a flow rate of 100 cm/hr. The column is then washed with 3 column volumes of 20% buffer B and 80% buffer A (see below). Insulin(B-30 threonine t-butyl ester-t-butyl ether) is then eluated by a linear gradient elution (increasing the percentage of buffer B from 20 to 50% in 15 column volumes). Elution begins at ˜30% B and ends at ˜40% B. The eluate is collected in appropriate size fractions, analyzed and pooled. Purity of >97% is achived. About 2.25 g of purified Insulin(B-30 threonine t-butyl ester-t-butyl ether) is obtained from 3 g loaded on to the column (75% yield). The column is regenerated with 100% B, followed by cleaning-in-place with 0.5N NaOH and finally stored in 20% 2-propanol till further use. Purified material from the chromatography eluate may be isolated by isoelectric precipitation and drying, and subjected to hydrolysis to obtain native insulin (see example 4).

### Chromatographic Conditions:

| | |
|---|---|
| Resin: | Amberchrome CG 300sd, 35 μm |
| Column: | Pharmacia XK 26 with a bed height of 30 cm |
| | (150 mL resin) |
| Eluent: | Buffer A: 10% v/v 2-propanol in water for injection, 0.1% |
| | TFA |
| | Buffer B: 80% v/v 2-propanol in water for injection water |
| | for injection, 0.1% TFA |
| Gradient: | linear from 20% B to 50% B in 15 column volumes |
| Flow rate: | 100 cm/h during load, 60 cm/h during elution |
| Temperature: | ambient temperature, approx. 20 to 25° C. |

### EXAMPLE 6

### Purification of Native Insulin on Amberchrome CG 300XT

200 mg of native insulin, generated by trifluoro acetic acid mediated hydrolysis of purified insulin-t-butyl ester-t-butyl ether obtained (see example 4 for hydrolysis and example 5 for purification) is dissolved in 100 ml water containing 10% 2-propanol. 10 mm×250 mm HPLC column is packed with Amberchrome CG300XT, regenerated with 0.5N NaOH, washed with water, and then 80% isopropyl alcohol containing 0.1% TFA. The column is equilibrated with buffer A (5CVs) and insulin solution applied at a flow rate of 100 cm/h. The column is then washed with 3 column volumes of 20% buffer B and 80% Buffer A (see below). Buffer B is then increased from 20% to 40% in one column volume and native insulin is eluated by a linear gradient of 40-50% of buffer B in 30 column volumes. Appropriate size fractions were collected, analyzed and pooled to generate 160 mg insulin (>98% pure).

| | |
|---|---|
| Resin: | Amberchrome CG300XT, 20 μm |
| Colunm: | 10 mm × 250 mm HPLC column packed with |
| | Amberchrome CG300XT |
| Eluent: | Buffer A: 10% v/v 2-propanol in 50 mM sodium sulfate, |
| | 2% acetic acid |
| | Buffer B: 50% v/v 2-propanol in 50 mM sodium sulfate, |
| | 2% acetic acid |
| Gradient: | linear from 40% B to 50% B in 30 column volumes |
| Flow rate: | 100 cm/h |
| Temperature: | ambient temperature, approx. 20 to 25° C. |

### EXAMPLE 7

### Purification of Insulin on Amberchrome CG 300XT

200 mg of native insulin generated by trifluoroacetic acid mediated hydrolysis of insulinB30(threonine)-t-butyl ester-t-butyl ether (obtained by transpeptidation, example 3, but without further purification, and then subjected to hydrolysis as described in see example 4), is dissolved in 100 ml water containing 10% 2-propanol. (10 mm×250 mm) HPLC column is packed with Amberchrome CG300XT regenerated with 0.5N NaOH, washed with water, followed by 80% isopropyl alcohol containing 0.1% TFA. The column is then equilibrated with buffer A (5 column volumes). Insulin solution was applied at a flow rate of 100 cm/h. The column is washed with 3 column volumes of 20% buffer B and 80% Buffer A (see below). Buffer B is then increased from 20% to 40% in one column volume and native insulin is eluated by a linear gradient of 40-50% of buffer B in 30 column volumes. Appropriate size fractions were collected, analyzed and pooled to generate 130 mg insulin (>98% pure).

| | |
|---|---|
| Resin: | Amberchrome CG300XT, 20 μm |
| Colunm: | 10 mm × 250 mm HPLC column packed with |
| | Amberchrome CG300XT |
| Eluent: | Buffer A: 10% v/v 2-propanol in 50 mM sodium sulfate, |
| | 2% acetic acid |
| | Buffer B: 50% v/v 2-propanol in 50 mM sodium sulfate, |
| | 2% acetic acid |
| Gradient: | linear from 40% B to 50% B in 30 column volumes |
| Flow rate: | 100 cm/h |
| Temperature: | ambient temperature, approx. 20 to 25° C. |

### FIG. 1

The expression vector used for the expression and secretion of Insulin polypeptide in the present invention. MOX-promoter refers to the alcohol inducible promoter methanol oxidase promoter, MOX-T refers to the methanol oxidase terminator. Amp refers to the amplicillin resistance conferring gene and URA3 is the yeast auxotropic selection marker.

1-63. (canceled)

64. A process for the isolation of insulin-like material from a solution comprising the insulin-like material and related impurities, by chromatography on reverse-phase poly-styrenic resin.

65. The process according to claim 64, wherein the resin is, polystyrene-divinylbenzene resin.

66. The process according to claim 64, wherein the solution containing insulin-like material and impurities are eluants of previous chromatographic steps.

67. The process according to claim 64, wherein the insulin-like material is a product of a chemical reaction.

68. The process according to claim 67, wherein the chemical reaction is a transpeptidation reaction of an insulin precursor polypeptide with an amino acid-ester-ether.

69. The process according to claim 68, wherein the insulin precursor polypeptide is des(B30)Insulin.

70. The process according to claim 68, wherein the amino acid ester-ether is an amino acid-alkyl ester-alkyl ether.

71. The process according to claim 70, wherein the amino acid-alkyl ester-alkyl ether is an amino acid-butyl-ester-butyl ether.

72. The process according to claim 71, wherein the amino acid-butyl ester butyl ether is amino acid-t-butyl ester-t-butyl ether.

73. The process according to claim 72, wherein the amino acid-t-butyl ester-t-butyl ether is threonine-t-butyl ester-t-butyl ether.

74. The process according to claim 64, wherein the insulin like material is insulinB30(threonine)-t-butyl ester-t-butyl ether.

75. The process according to claim 67, wherein the chemical reaction is a hydrolytic reaction.

76. The process according to claim 75, wherein the hydrolytic reaction is the hydrolysis of insulin(B30) ester-ether.

77. The process according to claim 76, wherein the insulin(B30) ester-ether is insulin(B30) alkyl ester-alkyl ether.

78. The process according to claim 77, wherein the insulin(B30) alkyl ester-alkyl ether is insulin(B30) butyl ester-butyl ether.

79. The process according to claim 78, wherein the insulin(B30) butyl ester-butyl ether is insulin(B30)-t-butyl ester-t-butyl ether.

80. The process according to claim 64, wherein the insulin-like material is native insulin.

81. A process for the isolation of insulin-like material from a solution comprising the insulin-like material and related impurities, by chromatography on reverse-phase poly-styrenic resin, the process comprising:
a) regenerating the resin under alkaline conditions;
b) equilibrating the resin in a water miscible organic solvent;
c) loading the resin with a solution comprising insulin like material;
d) eluting the resin in a linear gradient of a water miscible organic solvent; and
e) isolating the insulin-like material.

82. The process according to claim 81, wherein the resin is regenerated with sodium hydroxide and the water miscible organic solvent is an alcohol.

83. The process according to claim 82, wherein the alcohol is isoproponal.

84. The process according to claim 81, wherein the resin is equilibrated with about 80% isoproponal and the elution is carried out in a linear gradient of about 10% to about 80% isoproponal.

85. The process according to claim 84, wherein the linear gradient is from about 10% to about 50% isoproponal.

86. The process according to claim 81, wherein the insulin like material is insulinB30(threonine) ester-ether.

87. The process according to claim 86, wherein the insulinB30(threonine) ester-ether is insulinB30(threonine)-t- butyl ester-t-butyl ether.

88. The process according to claim 81, wherein the insulin like material is native insulin.


SEQUENCE LISTING (2 sequences)

SEQ ID NO: 1 - protein, 135 aa, Homo sapiens
MRFPSIFTAVLFAASSALAAPVNTTTEDETAQIPAEAVIGYSDLEGDFDVAVLPFSNSTNNGLLFINTTIASIAAKEEGVSLDKRFVNQHLCGSHLVEALYLVCGERGFFYTPKGIVEQCCTSICSLYQLENYCN

SEQ ID NO: 2 - DNA, 408 bp, Homo sapiens
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggataaaagatttgttaaccaacacttgtgtggctctcacttggtggaggcgttgtacttggtttgcggcgagcgtggtttcttctacactcctaagggcatcgttgagcaatgttgtacctccatctgttccttgtaccagctggagaactactgtaactga